(19) United States
(12) Patent Application Publication
(10) Pub. No.: US 2010/0317537 A1
(43) Pub. Date: Dec. 16, 2010

(54) BIOPHYSICAL PARAMETERS FOR SYSTEMS BIOLOGY

(75) Inventors: Yiider Tseng, Gainesville, FL (US); Jerry S.H. Lee, Bethesda, MD (US); Pei-Hsun Wu, Gainesville, FL (US)

Correspondence Address: SALIWANCHIK LLOYD & SALIWANCHIK;A PROFESSIONAL ASSOCIATION, PO Box 142950, GAINESVILLE, FL, 32614, US

(73) Assignee: University of Florida Research Foundation, Inc., Gainesville, FL (US)

(21) Appl. No.: 12/866,529

(22) PCT Filed: Feb. 13, 2009
(86) PCT No.: PCT/US09/34051

§ 371 Date: Aug. 6, 2010

Related U.S. Application Data
(60) Provisional application No. 61/029,097, filed on Feb. 15, 2008.

(51) Int. Cl. C40B 30/02; C12Q 1/02; C40B 30/06; G06F 17/30; G06F 17/10

(52) U.S. Cl. 506/8; 435/29; 506/10; 707/769; 703/2; 977/773; 977/904

(57) ABSTRACT

The invention relates to apparatus and methods for studying intracellular rheology. The invention further relates to use of such apparatus and methods to screen for potentially therapeutic molecules that give rise to rheological effects within a cell. As one example, the disclosed ballistic intracellular nanorheology (BIN) apparatus and methods may be employed in a high-throughput screen to identify mediators or inhibitors of the cytoskeletal modifications involved in cancer metastasis.

### CROSS-REFERENCE TO RELATED APPLICATION

This application claims the benefit of U.S. Provisional Application Ser. No. 61/029,097, filed Feb. 15, 2008, the disclosure of which is hereby incorporated by reference in its entirety, including all figures, tables and amino acid or nucleic acid sequences.

### BACKGROUND OF THE INVENTION

Cancer is a group of diseases characterized by uncontrolled growth and spread of abnormal cells. In 2007, it is estimated that more than 1.44 million new cases of cancer will be diagnosed in the United States, and more than 550,000 Americans will die of cancer. Cancer thus accounts for nearly 25% of all deaths in the U.S., second only to heart disease as the leading cause of death for Americans.

Cancer metastasis refers to a process by which cancerous cells are able to break away from a primary tumor and spread to other parts of the body. The ability to metastasize contributes greatly to the deadliness of cancer, and the prognosis for patients whose cancer has metastasized is typically grim relative to those whose disease is limited to a primary tumor. Survival of individual and isolated clusters of tumor cells dictates metastatic efficiency. Whereas normal epithelial cells in the body undergo programmed cell death if not attached to the extracellular matrix, metastasizing cancer cells acquire anchorage independence and thus remain viable as they are carried to distant locations within the body via the bloodstream or lymphatic system. Metastasizing cancers also exhibit amoeboid cell motility, an ability to move via the extension and retraction of cellular protuberances.

There is an urgent need for therapeutic agents capable of specifically inhibiting metastasis. Non-metastatic cancer is far more susceptible to treatment, and hence significant decreases in cancer mortality could be realized if metastasis could be inhibited.

### BRIEF SUMMARY OF THE INVENTION

The invention relates to methods for studying intracellular rheology and the use of such methods for the screening of candidate compounds. As one example, a ballistic intracellular nanorheology (BIN) apparatus and methods may be employed in a high-throughput screen to identify mediators or inhibitors of the cytoskeletal modifications involved in cancer metastasis or to screen candidate compounds for their effects on cytoskeletal modifications on malignant or cancerous cells.

### BRIEF DESCRIPTION OF THE DRAWINGS

FIG. 1 shows a schematic procedure of intracellular nanorheology.

FIGS. 2A-2D show kinetics of cytomechanical response of Swiss 3T3 cells to Rho activation. FIG. 2A shows time-dependent mean cellular compliances directly computed from the MSD of the thermal motions of nanospheres injected into the cytoplasm of serum-starved Swiss 3T3 fibroblast before and after treatment with 1 LPA. FIG. 2B shows time-dependent plateau modulus (represented by ) and Rho activities (represented by ▪) before and after the treatment of 1 μg/ml LPA. The Rho activities obtained from Western blots of Rho pull-down experiments (FIG. 2C). FIG. 2C shows a sample of Western blots from one Rho pull-down experiment. FIG. 2D shows time-dependent fluorescent micrographs of phalloidin, whereas focal adhesion (red) were visualized with a monoclonal antibody (mAb) against vinculin and Alexa 566 goat anti-mouse. Bar, 20 μm. Inset, magnified view of focal adhesions at the ends of actin stress fibers.

FIGS. 3A-3D show cytomechanical response of Swiss 3T3 cells to Rho activation and Rho kinase inhibition. FIG. 3A shows time-dependent mean cellular compliances directly computed from the MSD of the thermal motions of nanospheres injected into the cytoplasm of 10 μM Y-27632-treated, serum-starved Swiss3T3 cells before and after treatment with 1 μg/ml LPA. FIG. 3B shows time-dependent plateau modulus (represented by ) and Rho activities (represented by ▪) before and after the treatment of 1μg/ml LPA. The Rho activities obtained from Western blots of Rho pull-down experiments (FIG. 3C). FIG. 3C shows a sample of Western blots from one Rho pull-down experiment. FIG. 3D shows time-dependent fluorescent micrographs of phalloidin, whereas focal adhesions (red) were visualized with a mAb against vinculin and Alexa 566 goat anti-mouse. Bar, 20 μm. Inset, magnified view of focal adhesions at the ends of actin stress fibers.

FIG. 4 shows a flowchart of high-throughput intracellular nanorheology (integration of the image acquisition and analysis units).

FIGS. 5A-5D show that the mean square displacement (MSD) is correlated to the peak intensity (I′) of the corresponding microsphere tracked by a charge-coupled device (CCD) camera. A microsphere's peak intensity was estimated from the average intensity of each tracked object over all frames. FIG. 5A: A MSD vs. time lag plot of microspheres (n=47) embedded in glycerol shows the presence of MSD variation in a homogeneous aqueous solution (arrow head). The particle tracking experiments were conducted at a time resolution of 33 msec with using 25% of full power of illumination. FIG. 5B: A logarithm plot of MSD (τ=33 msec) vs. peak intensity of microspheres (n=53) embedded in glycerol under 25% (▴) and 100% (▪) power of illumination suggests a relationship between increasing peak intensity and decreasing MSD value. Data points acquired under 100% power of illumination are overall having higher peak intensity. The mean value of MSD (τ=33 msec) and the range of its standard deviation are shown by the error bar. FIG. 5C: Digital intensity signal (I_{PS}) and noise (I_{PN}) values are extracted from uniform light sources: the head light without a filter at various intensities (Δ), the head light with a red filter at various intensities (+), and the UV-visible light with a red filter at different concentration of Rhodamine B-tagged 70 kD Dextran (). The I_{PS}-I_{PN} relationship can be obtained from a 4th order polynomial fitting to those conditions. FIG. 5D: Signal-noise-ratio (SNR) vs. digital signal intensity may be determined by the curve fitting described in panel 1C to well-estimate the SNR as a function of the digital signal strength ranging between saturated signal (65535 arbitrary unit; i.e., au) and dark current (˜1500 au).

FIGS. 6A-6E show that the static error (2ε²), two times variance of the positioning random variable, can be estimated for different microsphere intensities by using simulated Gaussian beads as tracking objects. FIG. 6A: A flow diagram of how to estimate static error by Monte Carlo simulation. Simulated Gaussian particles with specified parameters can be used to represent fluorescent microspheres tracked using a CCD camera. First, a set of parameters for a Gaussian particle was assigned to simulate the image and then noise was added to mimic the experimental imaging conditions. This noised Gaussian particle image is then tracked to locate the center. FIG. 6B: The distribution of tracked positions from a static Gaussian particle can be revealed after running 600 noise independent trials. Using three different intensities of Gaussian beads (I=5,000 (blue, right panel upper), 10,000 (grey, right panel middle), and 50,000 (yellow, right panel lower)) with μₓ=μ_{y}=0, Rₐ=0.54 and I_{B}=3,000, different distribution patterns of tracked positions were generated incorporating pixel noise into the simulation (left panel). This demonstrates how the intensity profile of a microsphere will determine the tracking position error. Three histograms in the right panel indicate the distribution of the experimental position error, εₚ (the displacement between the experimental center and the true center, μₓ=μ_{y}=0). A Gaussian bead possessing a higher intensity will generate a smaller experimental error with a sharper distribution. FIG. 6C: Static error vs. assigned peak intensity (I) is plotted for three different Gaussian beads. The beads differ from one another with respect to the values of Rₐ and/or I_{B} FIG. 6D: LEFT: The subpixel position of a microsphere affects the size of positioning error. Six hundred simulations of each Gaussian bead located at three center positions (blue, (0, 0); red, (−0.25 , −0.25); and green, (−0.5 , −0.5)) in the lower-left quadrant of a pixel shows the distribution of the tracked center position of Gaussian beads (upper panel). RIGHT: When the histogram of 6,000 positioning error simulations for Gaussian beads located at the center of a pixel is set as a reference, the differences of count in the position error suggests that the positioning error increases when the simulated bead is further away from the center of a pixel (lower panel). FIG. 6E: The color in the diagram illustrates the correlation between the static error and the Gaussian particle center location within a pixel at a resolution of 0.01 pixels. The color bar indicates the size of the positioning error.

FIGS. 7A-7C demonstrate the method to relate extracted static error from simulated beads to experimental microsphere images. FIG. 7A: The left flow chart demonstrates the process of estimating static error from raw particle image. The process retrieves tracked parameters from a raw image, maps the adequate parameters to simulate experimental images with the complementary Gaussian particle, and applies Monte Carlo simulation to estimate the static error. The right flow chart shows the procedure used to map parameters for simulated Gaussian beads to match experimental tracked parameters. FIG. 7B: A 4th order polynomial equation can be adopted to describe the relationship between the radius of the simulated Gaussian bead, Rₐ, and the radius of tracked microsphere, Rₐ′, with perfect fitting (R²=1). This result is independent of the peak intensity, I, and background intensity, I_{B}. FIG. 7C: The Gaussian bead peak intensity (I) vs. the experimental peak intensity (I′), plotted for three different Gaussian bead radii, showing a linear correlation between I and I′. The plot also suggests that the correlation is independent of the pixel background since lines are overlaid at the same Rₐ despite having pixel backgrounds that are set differently

FIGS. 8A-8E show that static error can be well assessed and calibrated for the MSDs of microspheres embedded in glycerol. FIG. 8A: A sample of fixed microspheres is used to verify the estimated static error. Theoretically, the MSD of the fixed microspheres is approximately zero. Hence, the calculated values of the MSD from tracking a group of fixed microspheres can represent the spatial error generated from the experimental system. The individual microsphere's peak intensity is inversely proportional to the static error of experimental results (taken as the MSD of the fixed microspheres) in a logarithm scale. FIG. 8B: The logarithm of the experimental static error (MSD at 33 msec) is in agreement with the corresponding logarithm of the estimated simulated static error with a strong linear fit, R²=0.99. FIG. 8C: Raw MSD data from particle tracking under 25% power of illumination (n=47) reveals a degree of heterogeneity in the data, but raw MSD data (n=53) and its calibrated MSD both obtained under 100% power of illumination have a similar scale and trend as the calibrated MSD from low illumination (25%). FIG. 8D: Mean viscous modulus of glycerol from the raw and calibrated MSD at 25% and 100% power illumination respectively. The viscous modulus G″, are estimated at time lags of 33 msec. The viscous modulus of the raw MSD at 25% power of illumination is significant lower than the calibrated case or high illumination case. The dash line indicates the viscous modulus measured by a conventional rheometer and star denotes a different state from two-tailed t-tests within P<0.05. FIG. 8E: The illustration explains how errors generated from the video tracking instrument can affect the MSD result in the case of glycerol. Measured MSD is the culmination of noise-free MSD and static error. Here the noise-free means the information doesn't affect by the imaging noise during image acquisition.

FIGS. 9A-9C show that static error can be calibrated for the MSD of 100-nm carboxylated polystyrene particles embedded in MC3T3-E1 fibroblast cells under red-fluorescence. FIG. 9A: An image acquired from the CCD-camera. Red dots indicate the positions of microspheres within the frame. FIG. 9B: A MSD vs. time lag plot extracted from the cellular system (80 particles in 7 cells) implies subdiffusive particle motion at shorter lag times, indicating a range of local microenvironments that the microspheres are encountering. FIG. 9C: Using the correction method to subtract out the estimated static error in the system revealed a new MSD profile, which implies more diffusive particle motion throughout the cellular environment at short lag times.

### DETAILED DESCRIPTION OF THE INVENTION

Rheology is the study of the deformation and flow of matter, encompassing measurements of mechanical properties such as viscosity and elasticity, for example. Measurements of intracellular mechanical properties may be relevant to understanding or detecting a wide variety of cellular phenomena. For example, intracellular viscosity may affect diffusion rates for signaling molecules within a cell, thereby affecting realized rates of intracellular signal transduction. Also, rheological measurements may be employed to monitor cellular changes that are otherwise difficult or impossible to detect. For example, subtle aspects of cytoskeletal remodeling may be difficult to visualize via immunofluorescence, but may exhibit distinctive rheological signatures.

The microenvironment controls a cell's physiological events by providing extracellular biochemical and biophysical cues. When the microenvironmental conditions are well defined, the measured mechanical properties of the intracellular region are highly consistent. When one understands how intracellular mechanics correlate to a cell's behavior, one might predict a cell's activity from its intracellular mechanics.

To probe the intracellular mechanics, a novel technique has been developed, called ballistic intracellular nanorheology (BIN). In this technique, the trajectories of nanospheres which have been ballistically bombarded into the cytoplasm of individual cells are traced and analyzed. The BIN technique allows probing of the in situ intracellular mechanical properties of different cell lines under different extracellular stimuli Several characteristics make BIN unique: 1) it is a single cell and intracellular assay, thus the sensitivity of probes won't be affected by shear perturbation or spatial occupancy of the extracellular matrix; 2) it can measure intracellular mechanics either locally or globally; 3) it possesses high spatio-temporal resolution (5 nm to 10 nm and 33 ms, respectively); 4) the mechanical properties can be measured over a broad range of frequencies, simultaneously; 5) time-dependent viscoelastic response to extracellular stimuli can be monitored; and 6) the mechanical response to complex signaling pathways may be investigated.

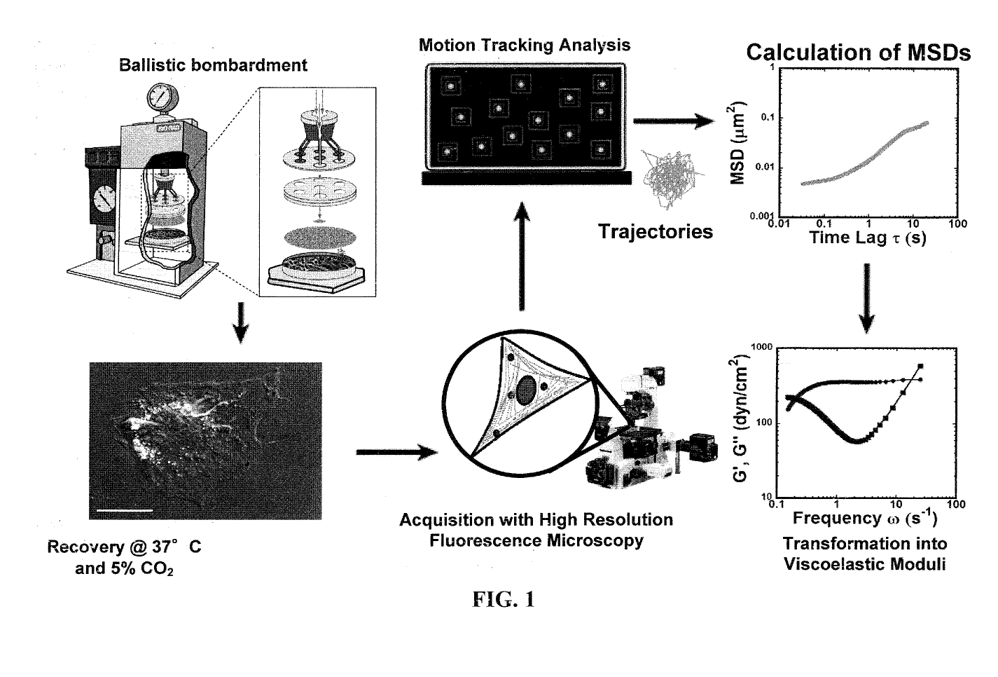

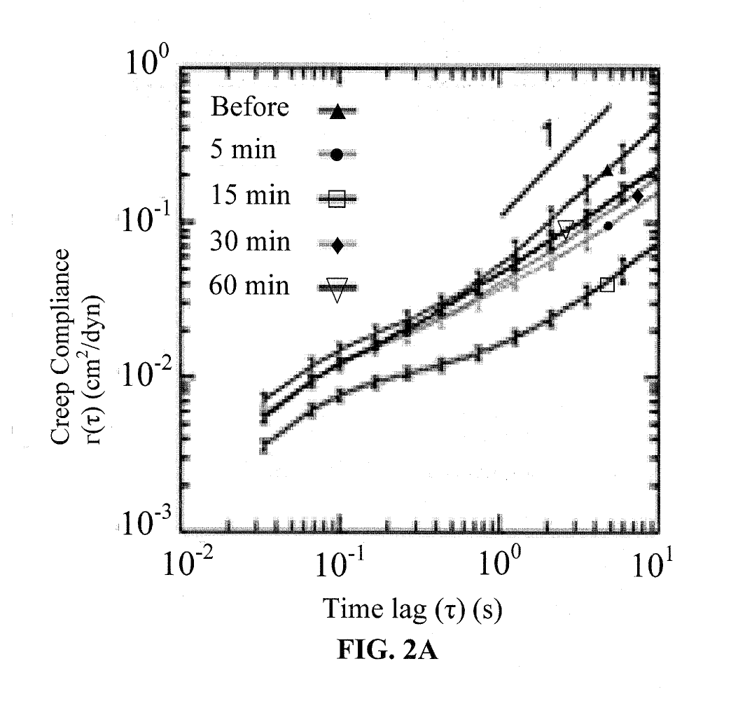

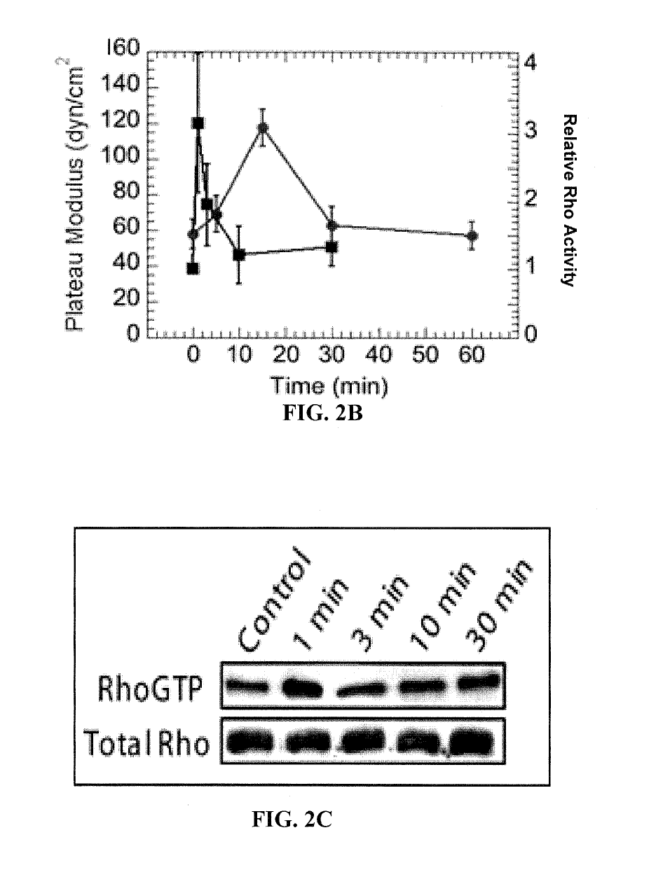

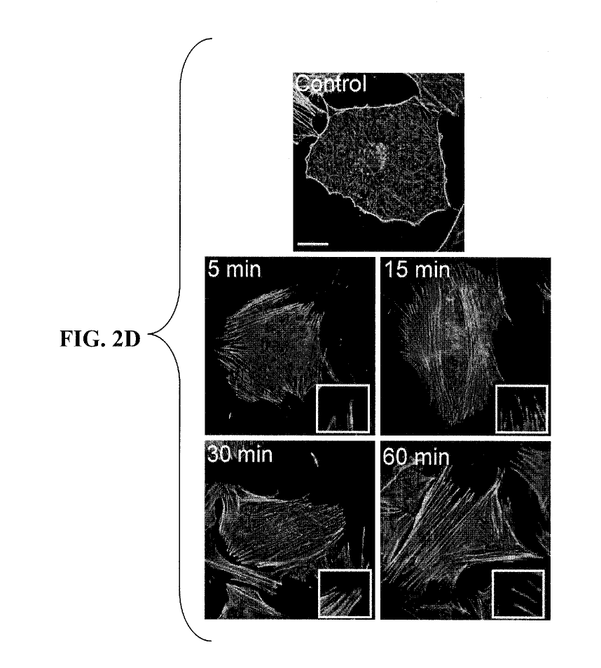

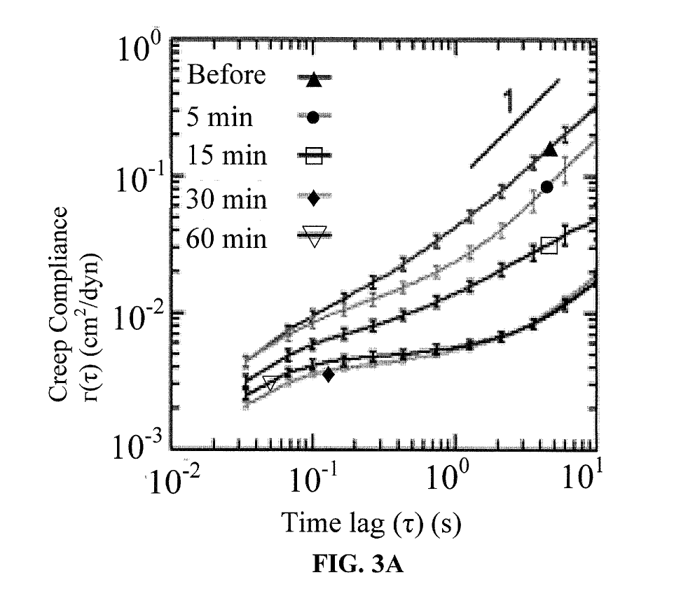

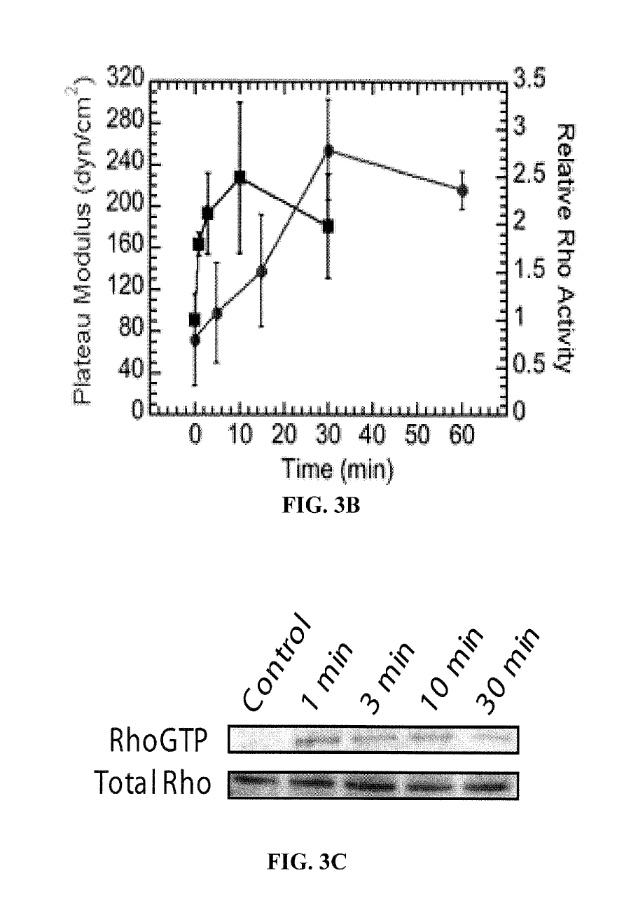

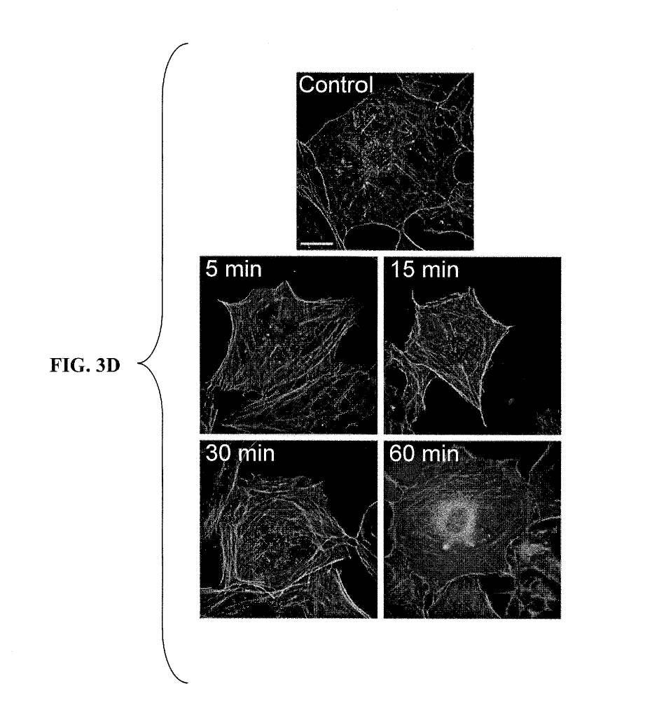

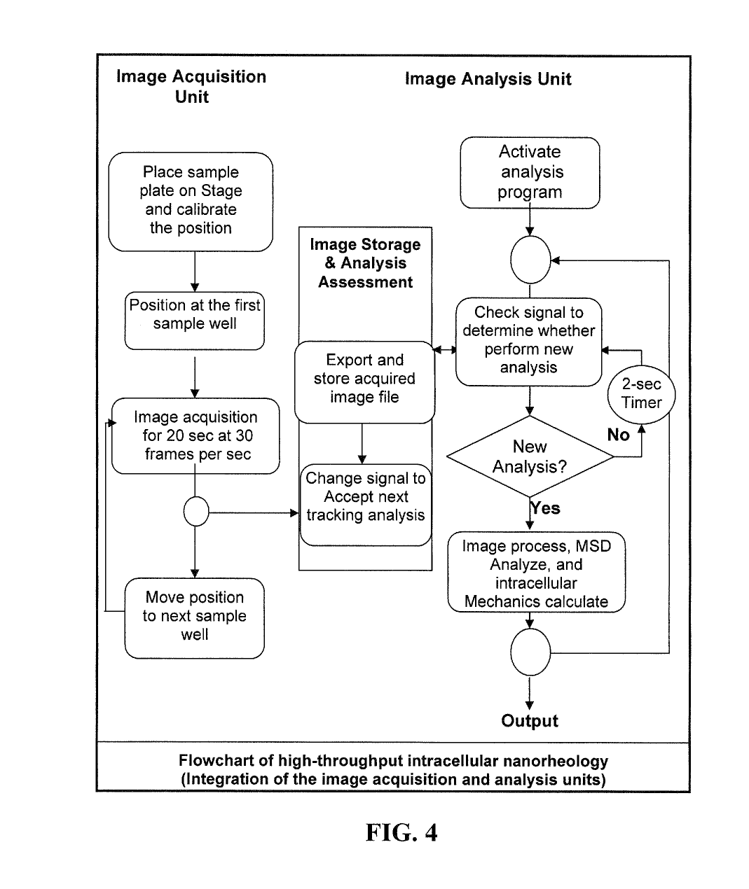

The current ballistic intracellular nanorheology (BIN) setup is introduced by its individual procedures in this section (the whole process of BIN is illustrated in FIG. 1). The first step in developing intracellular nanorheology was to introduce nanospheres into cells. In this step, nanospheres are introduced simultaneously to the entire tissue culture. For example, a Biolistic PDS-1000/He Particle Delivery System (Bio-Rad, Hercules, Calif.), originally designed to introduce DNA-coated microcarriers into cells for DNA delivery, can be used to deliver nanospheres into the cells of a tissue culture system. The ballistic bombardment is conducted in a sterilized chamber, which is further separated, by a divider, into upper and lower chambers. The divider is perforated in the center, where a rupture disk can be placed. When it is desired to deliver nanospheres into cells, cultured cells are placed on a 35 mm tissue culture disk in the bottom of the lower chamber, a nanosphere-coated rupture disk is placed on the divider to enclose the holes between the upper and lower chamber, and the chamber is sealed. Thereafter, the upper chamber is pressurized while the lower chamber is depressurized, creating a vacuum. After several seconds, the rupture disk is subjected to enough pressure difference to cause it to rupture; thus, the nanospheres are accelerated to very high speeds, shooting into the lower chamber. When these nanospheres are incident upon a tissue culture cell, they penetrate through cellular membrane and, due to their large momentum, enter the cytoplasm.

There are five operating parameters that control the success rate of ballistic bombardment; these include the upper chamber pressure, the lower chamber vacuum pressure, the pressure resilience of the rupture disk, the distance the nanosphere travels before hitting to cells, and the size of the nanosphere. The combination of these parameters determines the final penetrative momentum of the nanosphere into the cell membrane. In the table provided below, optimized parameters for several cell lines, such as Swiss 3T3 fibroblasts, mouse embryonic fibroblasts (MEF), HUVECs, Hela cells, and HCT-116 colon cancer cells have been provided. However, these parameters can be easily optimized for other cells lines or sources of cells (e.g., cancer or tumor cells obtained from a patient) and are not to be construed as limiting with respect to the delivery of ballistic nanoparticles/nanospheres into cells.

Exemplary Ballistic Bombardment Parameters

| | Helium Pressure | Vacumn | Target Distance |
|---|---|---|---|
| | (psi) | (torr) | (cm) |
| HCT116 | 1800 | 25 | 3 |
| Hela | 1800 | 25 | 3 |
| Swiss 3t3 | 1800 | 27.5 | 3 |
| MEF | 2200 | 27.5 | 3 |
| Huvec | 2200 | 27.5 | 3 |

The bombarded cells are then cultivated on a 35 mm tissue culture dish with a coverslip on the bottom (e.g., a glass coverslip). The dish is then mounted on an inverted epi-fluorescence microscope (Nikon, Melville, N.Y.) and maintained at 37° C. and 5% CO₂, for video acquisition (thermally excited motion can be analyzed to compute the local mechanical properties of the cytoskeletal network surrounding the particle). The fluctuating fluorescent nanospheres, embedded in the cytoplasm, can be examined using a 60-x, Plan Fluor oil-immersion objective with a numerical aperture 1.4 (Nikon) and video-recorded with a 16-bit Cascade 1K charge-coupled device (CCD) camera (Photometrics, Huntington Beach, CA). To track the nanosphere trajectories, video is collected onto a computer using microscope-camera-controlling software. Each frame of the video can be analyzed, using software, to calculate the centroid of the spheres in each frame. The displacements of the centroids of each particle will be monitored for 20 seconds (sec) at a frequency of 30 frames a second.

Images of nanospheres can be analyzed using the 2-D displacement of the centroid, for group of pixels, that contain the individual nanospheres' fluorescent signals (e.g., determined in the focal plane of the nanosphere for 20 sec at a rate of 30 frames per sec). The intensity-weighted centroid of the nanosphere can be tracked with 5 or 10 nm resolution, as determined by tracking the apparent displacement of a nanosphere rigidly attached to a coverslip. The density of nanospheres can be controlled at 10-30 nanospheres per field of view to reduce potential correlated interactions between neighboring nanospheres. At least 200 nanospheres should be tracked per condition for statistical purposes.

The 2-D displacements of an individual nanosphere, when analyzed from above, can be used to calculate the time-averaged mean square displacement (MSD), [symbol] Δr²(τ) [symbol] = [symbol] [x(t+τ)−x(t)]²+[y(t+τ)−y(t)]² [symbol] , where t is the elapsed time and r is the time lag, or time scale. Here, x and y are the time-dependent coordinates of the centroid of the nanosphere. To compute a time-averaged MSD, [symbol] Δr²(τ) [symbol] , it must be assumed that during the short time of movie capture (20 sec), no large change occurs in the micro-organization of the cell. Indeed, 20-sec is a much smaller time period when compared to the cell movement that one can document from cell migration (which usually takes hours). Cytoskeletal remodeling triggered by the Rho GTPase agonist, lysophosphatidic acid (LPA), also takes more than 10 minutes to finish. This time invariance means that, on average, the MSD between two time differences, of equal magnitude, is equal; for example, the MSD between 10 and 11 sec is equal to that between 11 and 12 sec. In this example, the time lag, τ, is 1 sec. The ensemble averaged MSD, <<Δr²(τ)>>, represents the mean MSD, which is equal to the sum of all measured MSDs divided by the number of tracked nanospheres.

The intracellular mechanics of cells can be represented by the elasticity and viscosity, denoted by G′(ω) and G″(ω), respectively. The G′(ω) indicates the immediate response of the regional cytoplasm and cytoskeleton to an applied force (or the energy storage capacity); the G″(ω) addresses the damping ability of the probed region (or the energy dissipation capacity). Studies in polymer physics and microfluidic mechanics have successfully developed a mathematical model to convert the MSD measurements of probed samples into their mechanical properties.

Considering the heterogeneous nature of the cellular cytoskeleton, the individual nanosphere probing results and the ensemble results are quantified and analyzed using statistical methods, such as analysis of variance (ANOVA) and student's t-test. After obtaining statistical means, the intracellular mechanics of cells under different conditions can be compared to address the effects and the cause.

Cell migration is a highly coordinated process, which is accomplished by precise cytoskeletal remodeling in a routine manner. It is known that cytoskeletal remodeling is governed by Rho GTPases (Rho, Rac, and Cdc42) in mammalian cells. The Rac and Cdc42 GTPases are collectively responsible for cell protrusion in the leading edge; meanwhile, Rho GTPase can induce the actomyosin contractile machinery and governs stress fiber formation in the trailing edge. Rho activities can be triggered by its agonist, LPA; and, upon Rho activation, the Rho signal can propagate in two distinct downstream pathways, the ROCK and mDia pathways. The Rho/mDia pathway is known to promote actin polymerization and promote filopodia formation, while the Rho/ROCK pathway mediates actomyosin contractility. Without both mDia and ROCK pathways being activated together, a cell cannot form stress fibers and, hence, cannot migrate. For example, Y-27632, a small molecule known to abolish ROCK pathway activity, can block stress fiber formation in cultured cells.

100-nm (or smaller) diameter nanospheres can be used for ballistic bombardment as set forth herein. These nanospheres (nanoparticles) can be luminescent. Optionally, the nanoparticles can be doped, selectively, with various luminescent dyes. Additionally, the surface of the luminescent nanoparticle can be modified with either carboxyl or polyethylene glycol groups as desired. The size of these nanoparticles can be controlled to as small as 30 nm. Thus, nanoparticles (luminescent or nonluminescent) of between 15 and 90 nm are specifically contemplated for use in the practice of the disclosed methods.

For different types of cells, cell morphologies and adhesion forces between cells and substrates are different. Thus, the cellular response to a pressure pulse generated during bombardment and to the vacuum pressure (even though exposed for less than 10 s) will depend on the cell type. For example, Swiss 3T3 fibroblasts can be ballistically bombarded using 2200-psi pressure for upper chamber, 27.5-torr vacuum for lower chamber, and 1-inch distance between rupture disk and culture dishes while the optimal Hela cells conditions are 1800-psi for upper chamber, 27-ton vacuum for lower chamber, and 1-inch distance between disk and dishes. A database can be constructed that provides the optimal parameters for other cell lines.

The image analysis portion of this technique can be executed using a subroutine incorporated into the commercially available software, such as MetaMorph. This subroutine can be designed to refresh the computer screen with updated data that is still being processed.

In one aspect of the invention, after bombarding, cells are cultivated on glass bottom 96-well plates for nanosphere tracking, such as poly-L-lysine coated glass bottom 96-well plates available from MatTek Corp. (Ashland, MA) or Nalge Nunc International (Rochester, N.Y.).

The automated, high-throughput BIN platform can also be used to systematically measure the effects of cytoskeletal related anti-cancer drugs, such as Y-27632 and/or 2,3-butanedione2-monoxime (BDM), on the intracellular mechanics. A significant intracellular mechanics change has been identified between the control and drug applied cells and it is possible to confirm the effect of various candidate compounds on various types of cancer cells by using the BIN platform as a reference for screening chemical compounds that can prevent the cytoskeletal remodeling triggered by agonists of cytoskeletal remodeling. These candidate compounds, which can prevent the cytoskeleton remodeling caused by the known agonist, can be further tested to verify their potential as anti-cancer drugs useful for the prevention of cell metastasis.

Thus, a screening method is envisioned in which a library of potential inhibitors of cytoskeletal remodeling are tested for activity. In one embodiment, a potential inhibitor would be administered to a test cell or cells but not to a control cell or cells. A known stimulus for cytoskeletal remodeling would then be applied to both the test cell(s) and the control cell(s). The stimulus could be, for example, an agonist or activator such as LPA, PDGF, or bradykinin, or a physical stimulus such as applied fluid shear. The response of the test cell(s) and control cell(s) would be monitored by ballistic intracellular nanorheology (BIN). Rheological responses indicative of inhibition of cytoskeletal remodeling in the test cell(s) relative to the control cell(s) would suggest that the tested potential inhibitor may be an actual inhibitor of cytoskeletal remodeling and may have efficacy as an anti-cancer drug.

The order of administration of the potential inhibitor and the stimulus for remodeling could be varied and it is envisioned that the screen would still be effective in identifying potential anti-cancer drugs. For example, the stimulus for cytoskeletal remodeling could be applied before, after, or concurrently with administration of the potential inhibitor of cytoskeletal remodeling.

In one aspect of the invention, processes associated with cancer metastasis may be monitored by BIN. For example, the processes involved in both anchorage independence and cell motility may be observable by their effects on local or global intracellular mechanical properties such as viscosity and elasticity. For example, cell motility is intimately associated with remodeling of the cytoskeleton, and such remodeling may be detectable via changes in intracellular mechanical properties. Likewise, anchorage independence may involve signaling pathways that begin with conformational changes at membrane-spanning integrins that bind to the extracellular matrix (ECM). Such integrins are associated intracellularly with the actin cytoskeleton and hence transduction of signals associated with cell attachment to the ECM may give rise to detectable mechanical effects associated with cytoskeletal perturbations.

Accordingly, certain aspects of the invention provide for methods of assessing candidate compounds for their effect on anchorage independence, cell motility and/or cytoskeleton remodeling. In such an aspect of the invention, cancerous or malignant cells (e.g., cells obtained from a cancer patient or known cell lines) are treated with a candidate compound and then observed for changes in anchorage independence, cell motility and/or cytoskeleton remodeling. Candidate compounds, in one aspect of the invention, can be known chemotherapeutic agents that are tested on malignant/cancer cells from a patient to determine those chemotherapeutic agents that would be useful for the treatment of the patient's cancer or malignancy. In a different aspect of the invention, candidate compounds can be obtained from compound libraries (e.g., proprietary compound libraries or publically available compound libraries (e.g., such as those available from the National Cancer Institute) and assessed for their activity on anchorage independence, cell motility and/or cytoskeleton remodeling of target cells (e.g., cancerous cell lines). Where the candidate agents are assessed for their effect upon a tumor or cancer cell, any candidate compounds that cause a decrease or reduction in the cell's viscosity or “stiffness” would be selected for further evaluation in compound development or for use in the treatment of a patient's cancer or malignancy.

### SELECTED EMBODIMENTS

### Embodiment 1

A method comprising inputting a designation of a cell type into a computer query and consequently receiving a set of experimental parameters recommended or required to be used for said cell type, ballistically introducing one or more nanoparticles into a cell of said cell type, observing the Brownian motion of at least one of the introduced nanoparticle(s), and calculating the value of an intracellular mechanical property based on said Brownian motion wherein:

said one or more nanoparticles have an average diameter of about 60 nanometers or less;

said calculating does not include refreshing a computer screen one time for every said one or more nanoparticles in every frame of a movie;

said calculating comprises using a computer algorithm to determine the position of the centroid of at least one of said one or more nanoparticles and said computer algorithm is selected from the group consisting of mass center algorithm, 2-D Gaussian fit by least square estimator algorithm, and simplex algorithm;

said computer algorithm is the algorithm that experimentally gives the most accurate results for the viscosity of one or more glycerin solutions when compared to results obtained for the same said one or more glycerin solutions when analyzed by conventional cone-and-plate rheometer; and multiple samples are analyzed by an automated or semi-automated process.

### Embodiment 2

The method of embodiment 1, wherein said automated or semi-automated process comprises cells being placed in a plurality of wells or other containers.

### Embodiment 3

The method of embodiment 1, wherein said observing and/or calculating comprise:

obtaining an experimental image of at least one of the introduced nanoparticle(s);

matching said experimental image to a corresponding simulated image; and

applying a correction factor based on said corresponding simulated image.

### Embodiment 4

A method of screening for anti-cancer therapeutic agents comprising administering to a cell a known mediator of cytoskeletal remodeling; administering to said cell a prospective therapeutic agent potentially capable of modifying the effect of said known mediator of cytoskeletal remodeling; analyzing said model cell by the method of embodiment 2; and comparing the results obtained for said cell to results obtained for a control cell.

### Embodiment 5

A method of screening for anti-cancer therapeutic agents comprising selecting a cell exhibiting a micromechanical property related to cancer virulence, contacting said cell with a prospective therapeutic agent (candidate compound) potentially capable of modifying said micromechanical property related to cancer virulence, and analyzing said cell by the method of embodiment 2 to determine whether said micromechanical property related to cancer virulence has been modified by said prospective therapeutic agent.

### Embodiment 6

A method of assessing candidate compounds for their effect on anchorage independence, cell motility and/or cytoskeleton remodeling of a cell comprising contacting a cell with a candidate compound and assessing the cell for a change in anchorage independence, cell motility and/or cytoskeleton remodeling, wherein said assessing is conducted via the method of embodiment 2.

### Embodiment 7

A method comprising ballistically introducing one or more nanoparticles into a cell, observing the Brownian motion of at least one of the introduced nanoparticle(s), and calculating the value of an intracellular mechanical property based on said Brownian motion, wherein said one or more nanoparticles have an average diameter of about 90 nanometers or less.

### Embodiment 8

The method of embodiment 7, wherein said one or more nanoparticles have an average diameter of about 60 nanometers or less.

### Embodiment 9

The method of embodiment 7, wherein said one or more nanoparticles have an average diameter of about 30 nanometers or less.

### Embodiment 10

The method of embodiment 7, 8, or 9, further comprising inputting a designation of a cell type into a computer query and consequently receiving a set of experimental parameters recommended or required to be used for said cell type.

### Embodiment 11

The method of embodiment 7, 8, 9, or 10, wherein said calculating does not include refreshing a computer screen one time for every said one or more nanoparticles in every frame of a movie.

### Embodiment 12

The method of embodiment 7, 8, 9, 10, or 11, wherein said calculating comprises using a computer algorithm to determine the position of the centroid of at least one of said one or more nanoparticles and wherein said computer algorithm is chosen from a set of algorithms consisting of mass center algorithm, 2-D Gaussian fit by least square estimator algorithm, and/or a simplex algorithm.

### Embodiment 13

The method of embodiment 12, wherein said computer algorithm is the algorithm that experimentally gives the most accurate results for the viscosity of one or more glycerin solutions when compared to results obtained for the same said one or more glycerin solutions when analyzed by conventional cone-and-plate rheometer.

### Embodiment 14

The method of embodiment 7, 8, 9, 10, 11, 12, or 13, wherein multiple samples are analyzed by an automated or semi-automated process.

### Embodiment 15

The method of embodiment 14, wherein the automated or semi-automated process comprises cells being placed in a plurality of wells or other containers.

### Embodiment 16

A method of screening for anti-cancer therapeutic agents comprising administering to a cell a known mediator of cytoskeletal remodeling; administering to said cell a prospective therapeutic agent potentially capable of modifying the effect of said known mediator of cytoskeletal remodeling; analyzing said model cell by the method of embodiment 7, 8, 9, 10, 11, 12, 13, 14, or 15; and comparing the results obtained for said cell to results obtained for a control cell.

### Embodiment 17

A method of screening for anti-cancer therapeutic agents comprising selecting a cell exhibiting a micromechanical property related to cancer virulence, contacting said cell with a prospective therapeutic agent (candidate compound) potentially capable of modifying said micromechanical property related to cancer virulence, and analyzing said cell by the method of embodiment 7, 8, 9, 10, 11, 12, 13, 14, or 15 to determine whether said micromechanical property related to cancer virulence has been modified by said prospective therapeutic agent.

### Embodiment 18

A method of assessing candidate compounds for their effect on anchorage independence, cell motility and/or cytoskeleton remodeling of a cell comprising contacting a cell with a candidate compound and assessing the cell for a change in anchorage independence, cell motility and/or cytoskeleton remodeling.

### Embodiment 19

The method of embodiment 18, wherein said assessing in conducted via the method of embodiment 7, 8, 9, 10, 11, 12, 13, 14, or 15.

### Embodiment 20

The method according to embodiment 16, 17, 18, or 19, wherein said cell is a cancerous or malignant cell.

### Embodiment 21

The method according to embodiment 16, 17, 18, or 19, wherein said candidate compound is a known chemotherapeutic agent and said cell or cells are cancerous or malignant cells obtained from a patient.

### Embodiment 22

The method according to embodiment 16, 17, 18, 19, 20, or 21, wherein said candidate compounds are obtained from compound libraries.

### Embodiment 23

The method according to embodiment 16, 17, 18, 19, 20, 21, or 22, wherein said candidate compounds are assessed for the ability to cause a decrease or reduction in the cell's viscosity.

### Embodiment 24

A method comprising ballistically introducing one or more nanoparticles into a cell, observing the Brownian motion of at least one of the introduced nanoparticle(s), and calculating the value of an intracellular mechanical property based on said Brownian motion, wherein said observing and/or calculating comprise:

obtaining an experimental image of at least one of the introduced nanoparticle(s);

matching said experimental image to a corresponding simulated image; and

applying a correction factor based on said corresponding simulated image.

### Embodiment 25

The method according to embodiment 7, 8, 9, 10, 11, 12, 13, 14, or 15, wherein said observing and/or calculating comprise:

obtaining an experimental image of at least one of the introduced nanoparticle(s);

matching said experimental image to a corresponding simulated image; and

applying a correction factor based on said corresponding simulated image.

### Embodiment 26

The method according to embodiment 16, 17, 19, 20, 21, 22, or 23, wherein said observing and/or calculating comprise:

obtaining an experimental image of at least one of the introduced nanoparticle(s);

matching said experimental image to a corresponding simulated image; and

applying a correction factor based on said corresponding simulated image.

### Example 1

### Improved Quantitative Cell Rheology By Combination of Experimental Data with Monte Carlo Simulations to Eliminate Inherent Static Error

Video-based particle tracking monitors the real-time motion of tracer particles. The mean square displacement (MSD) of these tracer particles may be used to interpret cellular biophysical properties, including the diffusivities of lipid membrane and transmembrane proteins, intracellular mechanics, and the dynamics of chromatin and nuclear bodies. Wieser et al., Biophys. J92, 3719-3728 (2007); Saxton & Jacobson, Annu. Rev. Biophys. Biomol, Struct. 26, 373-399 (1997); Lee et al., J. Cell Sci. 119, 1760-1768 (2006); Kole et al., Mol. Biol. Cell15, 3475-3484 (2004); Gorisch et al., Proc. Nat. Acad. Sci. U.S.A.101, 13221-13226 (2004); Jin et al., Biophys. 193, 1079-1088 (2007); Cabal et al., Nature 441, 770-773 (2006); Apgar et al., Biophys. J. 79, 1095-1106 (2000); Borgdorff & Choquet, Nature 417, 649-653 (2002); Haft & Edidin, Nature 340, 262-263 (1989). However, as more confined spaces are probed with higher temporal resolution, the ability of particle tracking to perform with consistent accuracy is diminished by the inherent measurement error. Martin et al., Biophys. J. 83, 2109-2117 (2002); Savin & Doyle, Biophys. J. 88, 623-638 (2005). For example, when imaging with a charge-coupled device (CCD) camera, the noise can fluctuate between individual pixels within tracking frames causing a positioning error. This error will be extended as static error to affect the accuracy of MSD analysis because the MSD is calculated from a particle's displacement, Savin & Doyle, Biophys. J. 88, 623-638 (2005); Thompson et al., Biophys. J. 82, 2775-2783 (2002); Cheezum et al., Biophys. J81, 2378-2388 (2001).

The characteristics of static error have been previously discussed from a theoretical perspective. Martin et al., Biophys. J. 83, 2109-2117 (2002); Savin & Doyle, Biophys. J. 88, 623-638 (2005); Thompson et al., Biophys. J. 82, 2775-2783 (2002). However, a method to precisely extract static error from individual experimental systems has not been known, and the accuracy of the MSD information used to decipher the biophysical properties of cellular systems has thus been limited.

In one aspect of the present invention, a new approach is used to accurately quantify static error. Using a Monte Carlo approach over a statistically meaningful number of trials, the standard deviation (the spatial resolution, ε) of the tracked positions of a static particle in an image was used as a quantitative measurement of the static error (2ε²). In this way, the dependence of static error on a particle's signal intensity, background intensity, radius, and center position within a pixel was individually quantified. Simulated images constructed from these controlling parameters were empirically mapped to experimental images so that the static error extracted from simulations could be applied to correct the MSD of actual experiments. An advantage of this strategy is that it solely relies on experimental outcomes, bypassing the details of complicated tracking algorithms and the various hardware specifications of tracking systems. More importantly, this method significantly improves the resolution of particle tracking experiments, greatly reducing ambiguities and potential errors in the interpretation of experiments.

The effectiveness of this approach was successfully tested by tracking particles in glycerol. Rheological measurements using this novel approach compare very well with conventional macroscopic rheological measurements. Additionally, creep compliance measurements in serum-starved MC3T3-E1 fibroblasts using this method revealed a greater degree of free diffusion than originally observed. In summary, this method offers a powerful approach for the significant advancement of particle tracking techniques used for microrheology.

### Results

### Light Source Affects the MSD Values

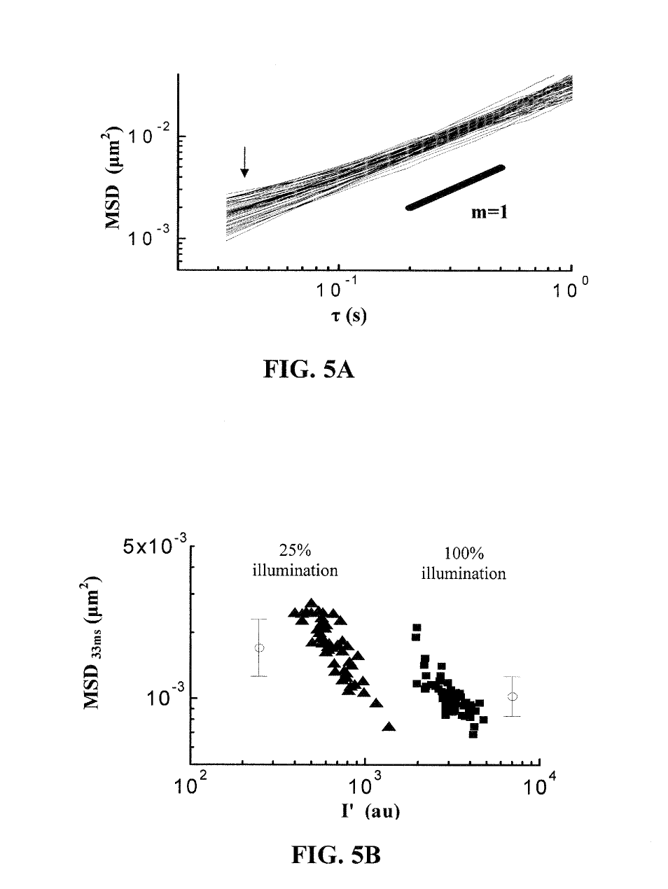

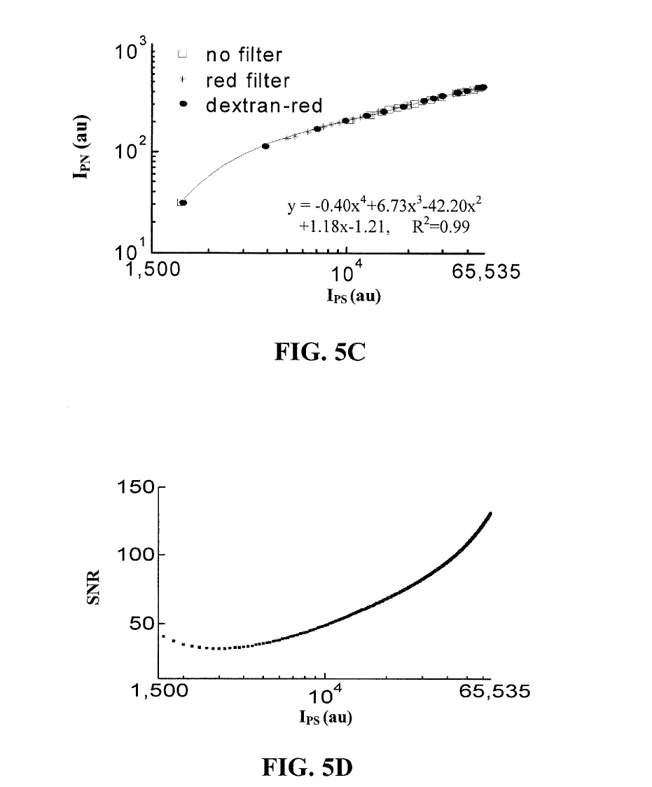

The consistency of a purely homogeneous medium should be reflected by identical MSD value for each tracked particle at any given time lag. This was not observed for glycerol, which had a distribution of MSD's inconsistent with a homogeneous medium, especially at shorter time lags (FIG. 5A). Analysis of this discrepancy revealed a correlation between MSD (τ=33 msec) and the peak intensity for individual microspheres (FIG. 5B). Emission outside of the microscope's focal plane or in the presence of microenvironmental heterogeneities may interfere with the light path from a microsphere to the photon detector, causing a distribution of peak intensity within a sample. Additionally, the digitization of photon signals by the detector introduces shot noise, and may also involve other types of noise. One aspect of the present invention relates to eliminating or mitigating the adverse effects of such suboptimal conditions, even when the cause or nature of the suboptimal conditions is not known or is incompletely known.

Subsequently, it was investigated whether the error revealed by the variation in MSD directly stems from the intensity fluctuations of the overall recorded signal. This was accomplished by extracting the signal and noise information from individual pixels throughout the whole image. Different pixels do not generate purely random noise under the same projected light due to noise inherent to the measurement device such as dark current variation and fixed pattern noise (Reibel et al., Eur. Phys. J. Appl. Phys. 21, 75-80 (2003)), which are consistently associated with an individual pixel and independent of outside signals. To eliminate this bias from each pixel, one reference image was set as a standard, and a successive image with the same illumination was then subtracted from the reference image. This procedure resulted in an even-weight (one bit of data per pixel) array with non-biased random noise. The random noise had an approximate Gaussian distribution and zero mean (consistently biased noise and the background intensity are filtered by the reference image subtraction). Therefore, the intensity of homogeneous light emitted from a halogen bulb can be determined by the mean pixel intensity (I_{PS}) for pixels over the whole image, and a distribution profile of random noise corresponding to the illumination source can be determined to obtain the mean random noise intensity (I_{PN}).

Using the above method, images of water were taken under a homogeneous field of collimated light from a halogen bulb, either with or without a 590-nm cut-off (red) filter in the light path, or with various concentrations of rhodamine B-labelled dextran with a red filter, to extract the I_{PS} and the I_{PN} particular to the microscope system being used. Using a CCD camera, a consistent I_{PS}-I_{PN} correlation emerged from each of the three different experimental settings, over the full working range of light intensity (FIG. 5C). Therefore, the correlation between I_{PS} and I_{PN} suggests that a tracking system could possess a digital output signal dependent noise, which cannot be simply expressed by only shot noise (I_{PN}=I_{PS}^{1/2}) (Cheezum et al., Biophys. J81, 2378-2388 (2001)), Gaussian noise (I_{PN}=N, where N is a constant) (Savin & Doyle, Biophys. J. 88, 623-638 (2005)), nor a combination of both (I_{PN}=I_{PS}^{1/2}+N) (Thompson et al., Biophys. J. 82, 2775-2783 (2002)).

Consequently, this information was used to effectively estimate the signal-to-noise ratio (I_{PS}/I_{PN}, or SNR) for pixels over the full spectrum of I_{PS} (FIG. 5D). These data further revealed that varying light intensity drastically affects the SNR for the camera readout, with brighter particles yielding better spatial resolutions. Furthermore, because the settings of a CCD camera (such as the gain in on-chip multiplication) can alter the correlation between I_{PS} and I_{PN}, the method demonstrated here offers a generic procedure to easily extract the SNR profile from any CCD camera-based tracking system for static error determination.

### Interplay of Several Factors Determines the Static Error

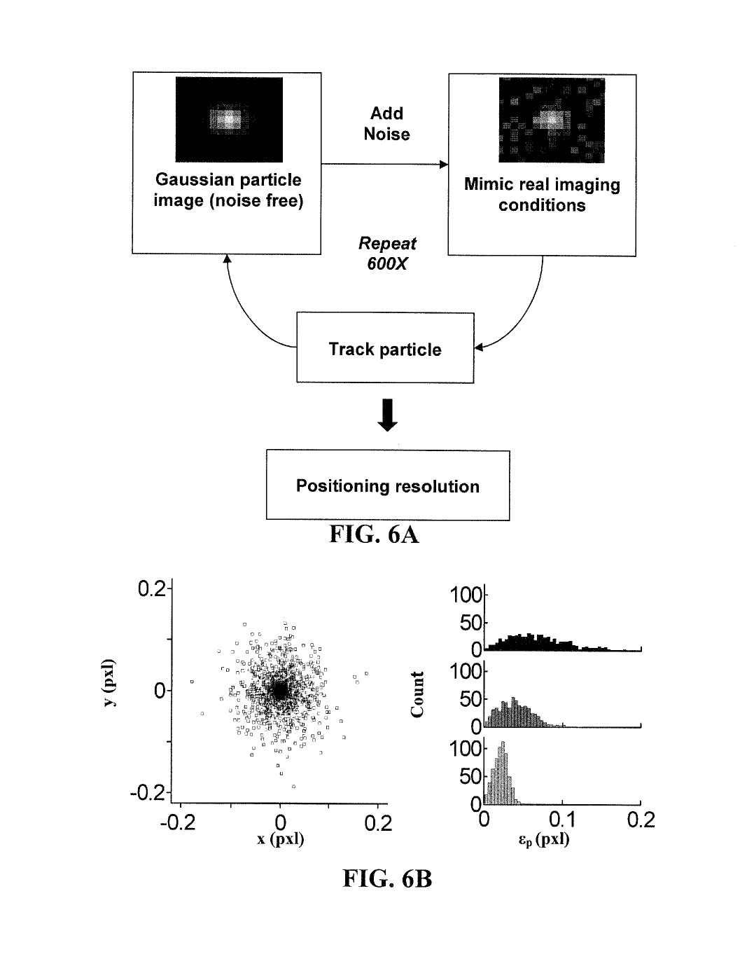

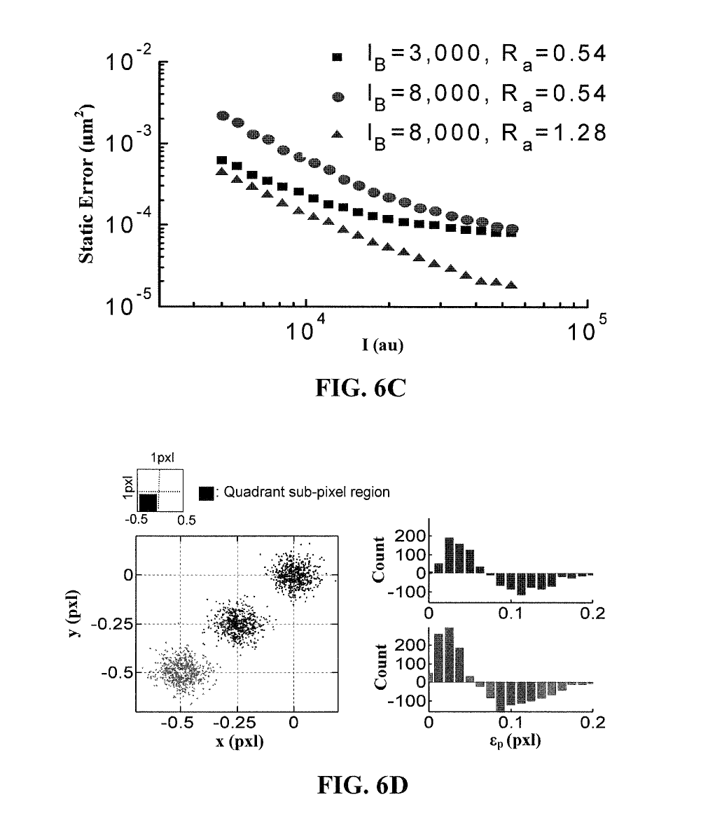

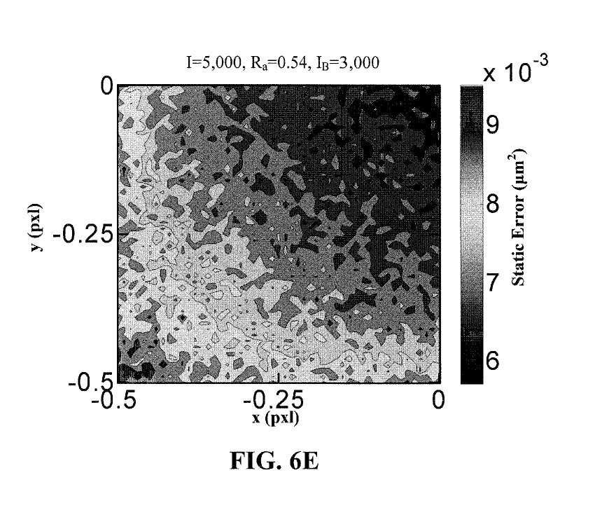

The SNR determined for the tracking system was then applied to create simulated images, which were used as a basis for investigating the conditions governing I_{PS} fluctuations and the degree of particle positioning bias. Several particle-tracking algorithms were examined (Savin & Doyle, Biophys. J88, 623-638 (2005); Cheezum et al., Biophys. J81, 2378-2388 (2001)), and a Gaussian algorithm was selected. A Gaussian-shaped simulated bead was constructed, which had a defined peak intensity (I), radius (Rₐ) and subpixel location (μₓ=μ_{y}=0 for the center of the pixel), with a homogeneous background intensity (I_{B}). Once the bead parameters were assigned, the appropriate level of random noise was added to individual pixels in the simulated image based on the established SNR (FIG. 5D). Subsequently, the simulated image containing the “system-noise” was added to the particle tracking argorithm to determine the “experimental” tracked position of the bead. These images were reconstructed multiple times to represent separate tracking trials under the given initial parameters, and the spatial resolution (i.e., standard deviation of the positioning distribution) of the bead was obtained after conducting a statistically meaningful number of such trials (FIG. 6A).

Using this Monte Carlo approach, an investigation was conducted of the relationship between the peak intensity of particles (I) and the resulting positioning distributions. Trials for three different Gussian bead peak intensities (μₓ=μ_{y}=0, Rₐ=0.54 and I=5,000, 10,000 and 50,000, respectively) with a uniform background intensity (I_{B}=3,000) suggested that the positioning error is related to the peak intensities (FIG. 6B, left). In addition, the brighter peak intensities resulted in a tighter distribution of tracked positions and a smaller positioning error (FIG. 6B, right). Since the spatial resolution (ε) can be quantitatively linked to the static error (2ε²), the brighter peak intensities directly translate to a diminished static error. Moreover, static error vs. the peak intensity was plotted for Gaussian beads having three sets of I_{B} and Rₐ values to demonstrate the dependence of static error on these additional parameters (FIG. 6C). In each case, the static error always decreased incrementally with Gaussian bead peak intensity.

The final Gaussian bead parameter that could have an effect on the static error profile was the subpixel location. Under a uniform I_{B}, Gaussian beads with a fixed I and Rₐ and were assigned different subpixel locations, i.e., (μₓ, μ_{y})=(0, 0), (−0.25 , −0.25) and (−0.5 , −0.5), where μᵢ=0 corresponded to the pixel center and μᵢ=−0.5 corresponded to the pixel edge, respectively. The static error extracted from the set centered within the pixel was used as a reference to observe deviations in the error distribution at other bead locations. Monte Carlo simulations suggested a trend of increasing error as Gaussian beads move closer to the pixel edge (FIG. 6D). To further understand this trend, the evaluation of sub-pixelation effects on the static error was repeated throughout a whole pixel quadrant (since there is symmetry about the pixel center in both the x- and y-axis). It was found that the subpixel position can augment static error up to 1.5 fold (from ˜6×10⁻³ μm² to ˜9×10⁻³ μm²) for a single set of assigned bead parameters (FIG. 6E). Thus, the sub-pixel localization of the bead center also contributes to the static error, revealing that several bead parameters collectively contribute to the propagation of such error.

### Direct Parameter-Mapping can be Used to Accurately Estimate the Static Error

Although the static error extracted from the Monte Carlo trials is affected by the individual manipulation of peak intensity, radius, subpixel location and background intensity values, these parameters may not be independent or constant throughout an actual experiment. As particles move out of the focal plane, their projected image will simultaneously appear to have a larger radius and a dimmer peak intensity than if they were in focus. The background intensity also changes for different microscopic and environmental conditions. Furthermore, some micro environments constrain particles so that the total displacement of a particle during short lag times can be less than the pixel size (i.e., a particle embedded in highly viscous and/or highly elastic media). In this case, subpixel localization of the particle will be a dominant factor for static error in the tracking analysis. Therefore, the accurate representation of experimental particles necessitates a case by case assignment of the proper Gaussian bead parameters to validate the Monte Carlo approach of extracting the spatial resolution using simulated images.

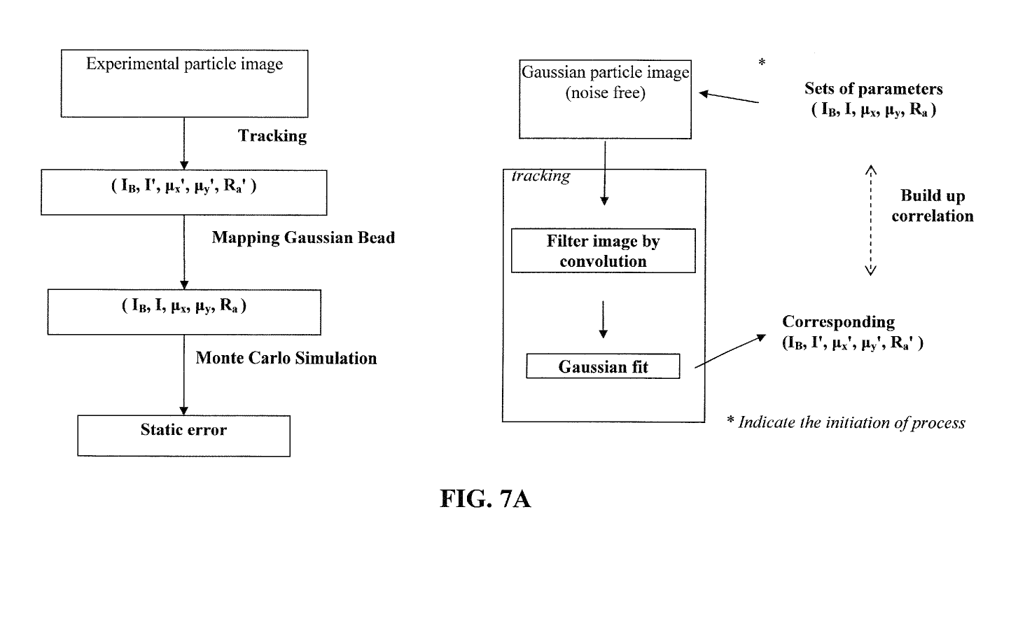

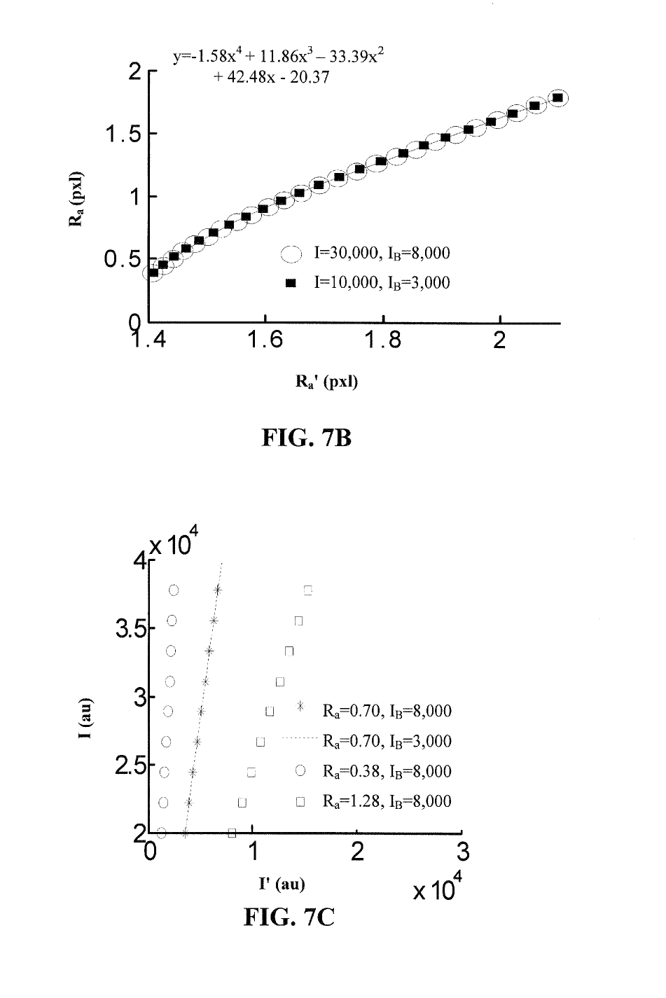

Particle tracking algorithms independently process microspheres in the acquired images and produce a set of experimental parameters, (Rₐ, I′, μₓ′ and μ_{y}′) to describe each tracked microsphere. However, these parameters cannot represent the true characteristics of particles because they have been processed by convolution of the tracking algorithm, and cannot be directly used to extract the static error by Monte Carlo simulation. A novel mapping procedure has been developed to estimate the true parameters (Rₐ, I′, μₓ′ and μ_{y}′) of the original microsphere from the convolved images of the non-linear algorithm tracking analysis (FIG. 7A). During this process, the addition of extracted system noise to the simulated images was omitted in order to avoid generating additional variation in the image data that would only corrupt the comparisons.

The mapping begins by assuming that the absolute position of a simulated Gaussian bead, μₓ, μ_{y}), is the same as the experimentally tracked positions, μₓ′, μ_{y}′). This assumption has previously been evaluated with the conclusion that the pixilization effects can only generate up to 0.02 pixels of error. Savin & Doyle, Biophys. J. 88, 623-638 (2005). Several simulated Gaussian bead images generated by a series of Rₐ values (from 0.38 to 1.80 pixels) and different peak/background intensities were subjected to the tracking algorithm to retrieve the corresponding apparent radii (Rₐ′). A scatter plot of Rₐ to Rₐ′ fit by a 4^{th}-order polynomial with perfect regression (R²=1) (FIG. 7B) is evidence that the Rₐ−Rₐ' correlation depends only on the tracking algorithm and is independent of the peak intensity of the Gaussian bead and the background pixel intensity. Having accounted for all other Gaussian bead parameters, the relationship between I and I′ was uncovered using a linear curve fitting (FIG. 7C). The entire mapping procedure was repeated for a range of Gaussian bead parameter configurations until a clear link between simulated and experimental tracking images was evident. Through this simple process, any typical microsphere experimental image can be precisely simulated by a corresponding Gaussian bead image.

### Procedure Verification Using In Vitro and In Situ Experimental Systems

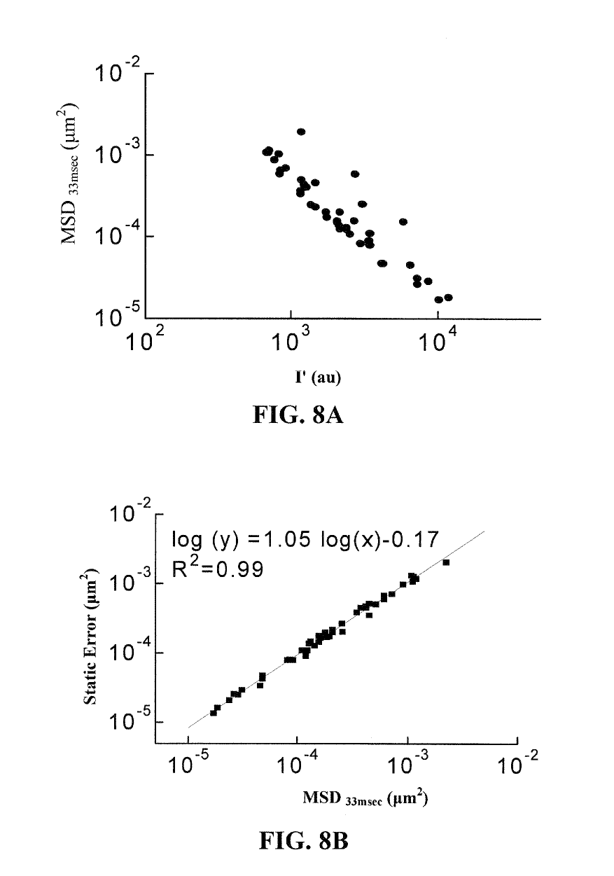

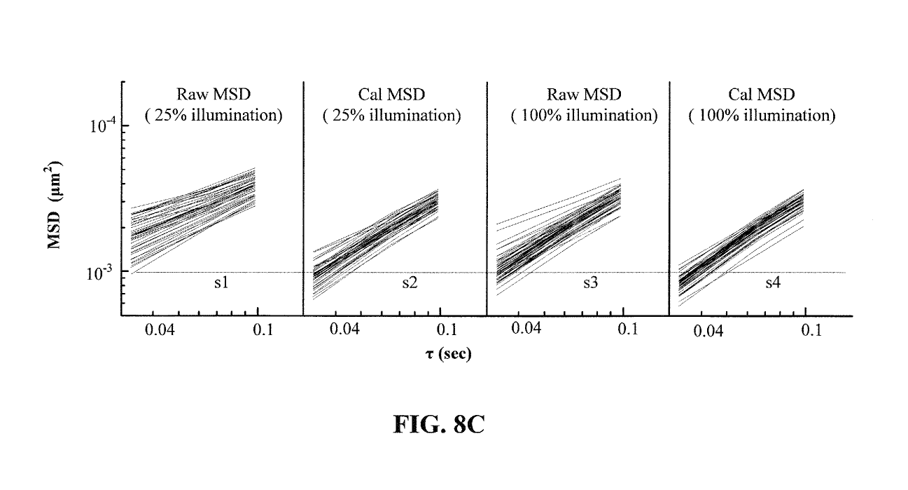

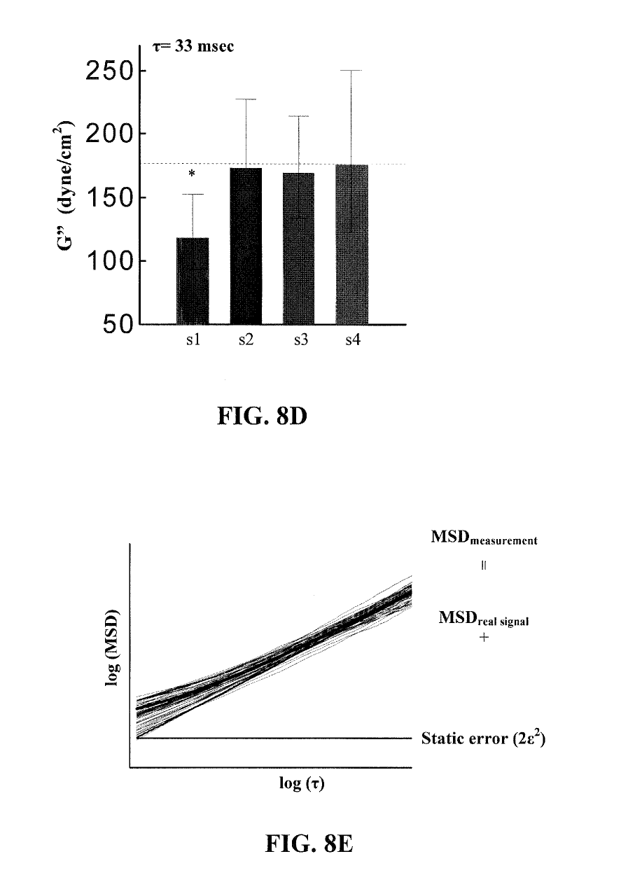

The accuracy of the mapping procedure was verified by imaging static particles. Several microspheres were immobilized onto a coverslip and their MSDs were tracked. Immobilized microspheres should exhibit approximately no movement, and the detected MSD values are expected to represent the static error. The mapping procedure was applied to estimate the static error from the experimental images. Comparing the experimental static error of each microsphere to its peak intensity revealed that static error invariably reduces when the peak intensity of the corresponding microsphere increases (FIG. 8A). Using the Monte Carlo simulation trials, the static error (2ε²) was extracted and correlated to the experimental static error in a log-log plot showing that the simulated static error is in agreement with the experimental results (MSD), having a strong linear correlation (R²=0.99) (FIG. 8B). This strong correlation confirms that the Monte Carlo simulation approach explained herein can successfully estimate real-time static error.

MSD data from a standard tracking analysis in glycerol was corrected using this technique by directly subtracting the estimated static error value. Comparison between the raw and corrected results under low (25%) and high (100%) illumination suggests that the correction produce significantly more precise results, reflecting the true nature of the homogeneous Newtonian fluid (FIG. 8C). When the generalized Stokes-Einstein Relation was used to convert the MSDs to the viscous modulus, it was found that the values are underestimated in the raw MSDs of low illumination, but are accurate when the MSDs are calibrated or are obtained from high illumination experiments (FIG. 8D). This provides another validation of the fact that static error is important in tracking experiments, and should be eliminated using the correction algorithm (FIG. 8E).

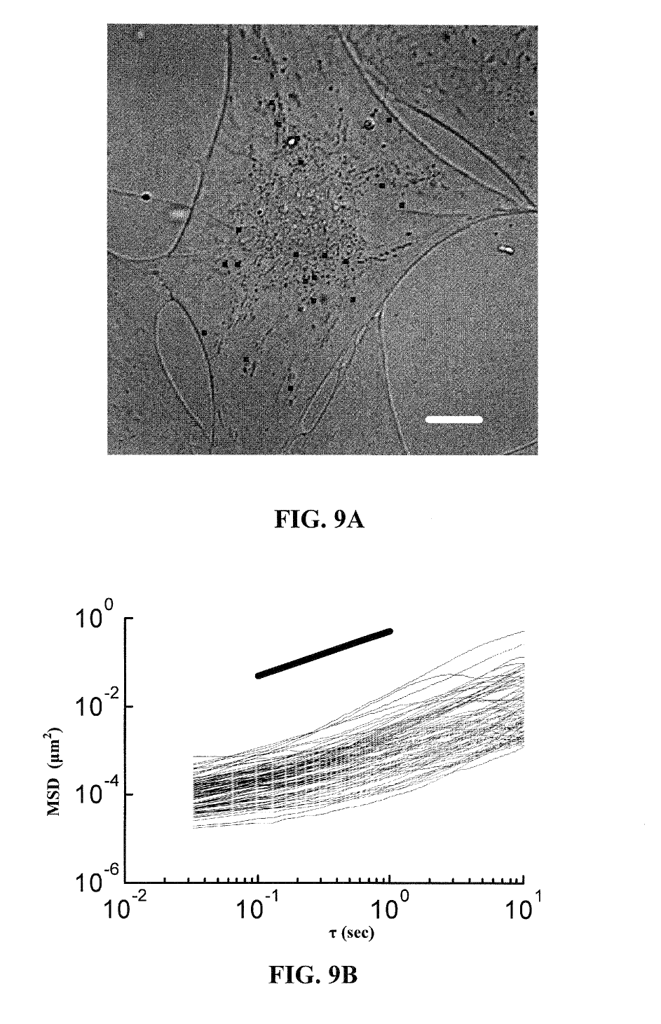

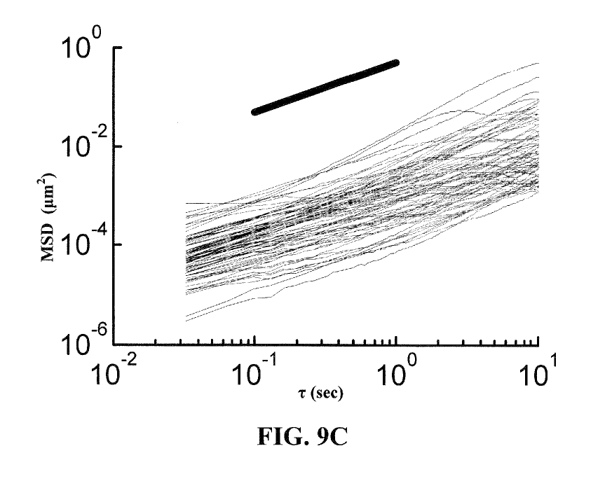

Further investigations demonstrated use of the correction (i.e., calibration) technique for tracking the positions of particles inside cells and calculating the creep compliance from the MSD data. Red fluorescent, carboxylated microspheres of 100-nm diameter were ballistically bombarded into the cytoplasm of several serum-starved MC3T3-E1 fibroblasts (FIG. 9A). Serum-starved cells lack major cytoskeletal structures such as the actin cytoskeleton, which may physically interfere with a particle's free diffusion. Therefore, inert microspheres embedded within such cells should exibit relatively free motion. After video-tracking using the conventional approach, the MSD profiles extracted from the movements of the fluorescent particles indicate that they move subdiffusively in the cytoplasmic region of the cells, contradicting what would be expected from these cells (FIG. 9B). However, the corrected MSD values obtained by the current approach suggest that these particles are actually much less subdiffusive than was previously measured (FIG. 9C). Lee et al., J. Cell Sci. 119, 1760-1768 (2006). This analysis clearly demonstrates the necessity of eliminating static error from particle tracking measurements, which can otherwise seriously bias conclusions about the physical properties measured using microrheology.

### DISCUSSION

MSD inaccuracy due to static error is ubiquitous in CCD camera-based particle tracking systems. However, the complex interplay between multiple tracking parameters had precluded the development of a practical method to minimize the errors. The correction (i.e., calibration) approach now explained herein significantly minimizes static error. This approach circumvents the complication of direct static error calculation by employing a simulation-based method to correct experimental particle tracking measurements. This considerably enhances the accuracy of the MSD and improves the subsequent estimation of diffusivity as well as rheological properties.

Conventional tracking of particles in a homogenous glycerol solution resulted in a wider MSD distribution at short lag times with decreasing light source intensity. This result indicates that static error can significantly bias the MSD profile, causing a serious misinterpretation of the underlying physical properties. Static error in the tracking system used herein can be estimated to be between ˜2×10⁻⁵ μm² and ˜10⁻³ μm² by tracking immobilized microspheres, suggesting that measured MSD values within this range are clearly unreliable. However, elimination of this static error allows for an accurate MSD measurement with a resolution less than 10⁻⁴ μm². Moreover, this correction technique is not limited to the particular system used herein, but is broadly applicable to any tracking system. The transition to another system requires simple steps of determining the correlation between the pixel signal and noise, and appropriately selecting correct tracking parameters. By following the methodology described herein, static error can be significantly eliminated, leading to a greater clarity when interpreting the MSD values from a particle tracking experiment.

All patents, patent applications, provisional applications, and publications referred to or cited herein, supra or infra, are incorporated by reference in their entirety, including all figures and tables, to the extent they are not inconsistent with the explicit teachings of this specification.

It should be understood that any examples and embodiments described herein are for illustrative purposes only and that various modifications or changes in light thereof will be suggested to persons skilled in the art and are to be included within the spirit and purview of this application.

1-26. (canceled)

27. A method comprising inputting a designation of a cell type into a computer query and consequently receiving a set of experimental parameters recommended or required to be used for said cell type, ballistically introducing one or more nanoparticles into a cell of said cell type, observing the Brownian motion of at least one of the introduced nanoparticle(s), and calculating the value of an intracellular mechanical property based on said Brownian motion wherein:
said one or more nanoparticles have an average diameter of about 60 nanometers or less;
said calculating does not include refreshing a computer screen one time for every said one or more nanoparticles in every frame of a movie;
said calculating comprises using a computer algorithm to determine the position of the centroid of at least one of said one or more nanoparticles and said computer algorithm is selected from the group consisting of mass center algorithm, 2-D Gaussian fit by least square estimator algorithm, and simplex algorithm;
said computer algorithm is the algorithm that experimentally gives the most accurate results for the viscosity of one or more glycerin solutions when compared to results obtained for the same said one or more glycerin solutions when analyzed by conventional cone-and-plate rheometer; and
multiple samples are analyzed by an automated or semi-automated process.

28. The method according to claim 27, wherein said automated or semi-automated process comprises cells being placed in a plurality of wells or other containers.

29. The method according to claim 27, wherein said observing and/or calculating comprise:
obtaining an experimental image of at least one of the introduced nanoparticle(s);
matching said experimental image to a corresponding simulated image; and
applying a correction factor based on said corresponding simulated image.

30. A method comprising ballistically introducing one or more nanoparticles into a cell, observing the Brownian motion of at least one of the introduced nanoparticle(s), and calculating the value of an intracellular mechanical property based on said Brownian motion, wherein said one or more nanoparticles have an average diameter of about 90 nanometers or less.

31. The method according to claim 30, wherein said one or more nanoparticles have an average diameter of about 60 nanometers or less.

32. The method according to claim 30, wherein said one or more nanoparticles have an average diameter of about 30 nanometers or less.

33. The method according to claim 30, further comprising inputting a designation of a cell type into a computer query and consequently receiving a set of experimental parameters recommended or required to be used for said cell type.

34. The method according to claim 30, wherein said calculating does not include refreshing a computer screen one time for every said one or more nanoparticles in every frame of a movie.

35. The method according to claim 30, wherein said calculating comprises using a computer algorithm to determine the position of the centroid of at least one of said one or more nanoparticles and wherein said computer algorithm is chosen from a set of algorithms consisting of mass center algorithm, 2-D Gaussian fit by least square estimator algorithm, and/or a simplex algorithm.

36. The method according to claim 35, wherein said computer algorithm is the algorithm that experimentally gives the most accurate results for the viscosity of one or more glycerin solutions when compared to results obtained for the same said one or more glycerin solutions when analyzed by conventional cone-and-plate rheometer.

37. The method according to claim 30, wherein multiple samples are analyzed by an automated or semi-automated process.

38. The method according to claim 37, wherein the automated or semi-automated process comprises cells being placed in a plurality of wells or other containers.

39. The method according to claim 30, wherein said observing and/or calculating comprise:
obtaining an experimental image of at least one of the introduced nanoparticle(s);
matching said experimental image to a corresponding simulated image; and
applying a correction factor based on said corresponding simulated image.

40. A method of screening for anti-cancer therapeutic agents comprising administering to a cell a known mediator of cytoskeletal remodeling; administering to said cell a prospective therapeutic agent potentially capable of modifying the effect of said known mediator of cytoskeletal remodeling; analyzing said model cell by the method of claim 30; and comparing the results obtained for said cell to results obtained for a control cell.

41. The method according to claim 40, wherein said cell is a cancerous or malignant cell.

42. The method according to claim 40, wherein said candidate compound is a known chemotherapeutic agent and said cell or cells are cancerous or malignant cells obtained from a patient.

43. The method according to claim 40, wherein said candidate compounds are obtained from compound libraries.

44. The method according to claim 40, wherein said candidate compounds are assessed for the ability to cause a decrease or reduction in the cell's viscosity.

45. A method of screening for anti-cancer therapeutic agents comprising selecting a cell exhibiting a micromcchanical property related to cancer virulence, contacting said cell with a prospective therapeutic agent (candidate compound) potentially capable of modifying said micromechanical property related to cancer virulence, and analyzing said cell by the method of claim 30 to determine whether said micromechanical property related to cancer virulence has been modified by said prospective therapeutic agent.
